(19) United States
(12) Patent Application Publication
(10) Pub. No.: US 2019/0290245 A1
(43) Pub. Date: Sep. 26, 2019

(54) ULTRASOUND OBSERVATION DEVICE, METHOD OF OPERATING ULTRASOUND OBSERVATION DEVICE, AND COMPUTER READABLE RECORDING MEDIUM

(71) Applicant: OLYMPUS CORPORATION, Tokyo (JP)

(72) Inventor: Takehiro YOSHIMURA, Tokyo (JP)

(73) Assignee: OLYMPUS CORPORATION, Tokyo (JP)

(21) Appl. No.: 16/383,728

(22) Filed: Apr. 15, 2019

Related U.S. Application Data
(63) Continuation of application No. PCT/IB2018/057912, filed on Oct. 12, 2018.

(30) Foreign Application Priority Data
Oct. 5, 2017 (JP) 2017-195391

(51) Int. Cl. A61B 8/08; A61B 8/00

(52) U.S. Cl. CPC A61B 8/5207; A61B 8/14; A61B 8/469; A61B 8/5269

(57) ABSTRACT

An ultrasound observation device includes a processor including hardware, the processor being configured to execute: setting any one of processing modes regarding multiple different processes performed on an ultrasound image by a user; performing, based on a command signal output from a touch-pad in accordance with a change in a touch position of a touching object, a process corresponding to the set processing mode, the ultrasound observation device; and determine, based on the set processing mode, a control to be performed out of a first control to stop a process on the ultrasound image and a second control to perform a predetermined process on the ultrasound image when it is detected that the touching object moves away from the touch-pad.

### CROSS-REFERENCE TO RELATED APPLICATION

This application is a continuation of International Application No. PCT/IB2018/057912, filed on Oct. 12, 2018, the entire contents of which are incorporated herein by reference.

### BACKGROUND

The present disclosure relates to an ultrasound observation device, a method of operating the ultrasound observation device, and a computer readable recording medium.

Ultrasound images generated by using ultrasound waves are sometimes used to examine or observe the property of living tissue or a material that is the observation target. An ultrasound observation device executes image processing on ultrasound signals that come from living tissue and received by an ultrasound endoscope to generate ultrasound images. In recent years, input devices using a touch-pad have been known to input the setting of a processing mode, an observation condition, or the like, to an ultrasound observation device (for example, see Japanese Laid-open Patent Publication No. 2008-136701 and Japanese Laid-open Patent Publication No. 2016-220830).

### SUMMARY

According to one aspect of the present disclosure, there is provided an ultrasound observation device including a processor including hardware, the processor being configured to execute: setting any one of processing modes regarding multiple different processes performed on an ultrasound image by a user; performing, based on a command signal output from a touch-pad in accordance with a change in a touch position of a touching object, a process corresponding to the set processing mode, the ultrasound observation device; and determine, based on the set processing mode, a control to be performed out of a first control to stop a process on the ultrasound image and a second control to perform a predetermined process on the ultrasound image when it is detected that the touching object moves away from the touch-pad.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a block diagram that illustrates a configuration of an ultrasound diagnosis system including an ultrasound observation device according to a first embodiment;

FIG. 2 is a diagram that illustrates a configuration of an input device illustrated in FIG. 1;

FIG. 3 is a flowchart that illustrates a process of the ultrasound observation device according to the first embodiment;

FIG. 4 is a block diagram that illustrates a configuration of an ultrasound diagnosis system including an ultrasound observation device according to a second embodiment;

FIG. 5 is a flowchart that illustrates a process of the ultrasound observation device according to the second embodiment;

FIG. 6 is a diagram that illustrates a behavior for performing a flick operation on the touch-pad;

FIG. 7 is a diagram that illustrates a process when a flick operation is input to the touch-pad;

FIG. 8 is a diagram that illustrates a behavior for performing a slide operation on the touch-pad;

FIG. 9 is a diagram that illustrates a process when a slide operation is input to the touch-pad; and

FIG. 10 is a diagram that illustrates an example of the screen when the second control is being performed.

### DETAILED DESCRIPTION

With reference to the drawings, embodiments are explained below. The present disclosure is not limited to the embodiments. The present disclosure is applicable to typical ultrasound observation devices, methods of operating an ultrasound observation device, and programs for operating an ultrasound observation device.

Moreover, in description of drawings, the same or equivalent components are attached with the same reference numeral as needed. Moreover, it should be noted that the drawings are schematic and the relation between elements in dimension, the ratio between elements, and the like, sometimes differ from reality. Each of the drawings sometimes contains parts that are different in dimensional relation or proportion.

### First Embodiment

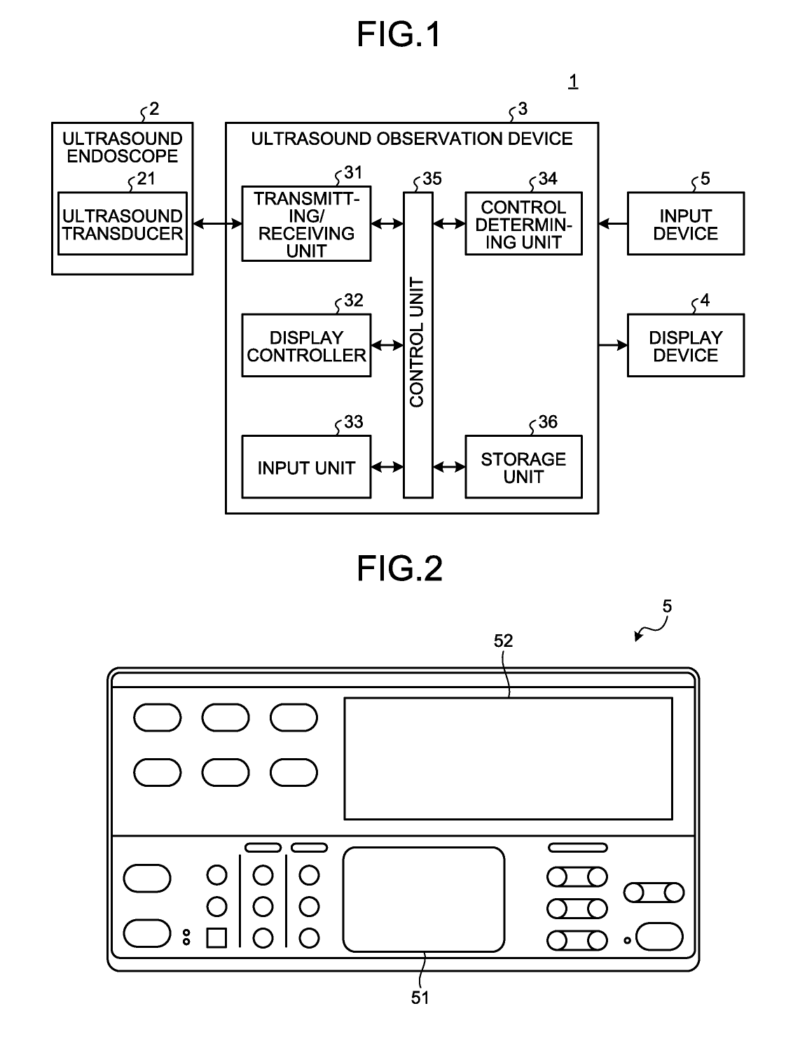

FIG. 1 is a block diagram that illustrates a configuration of an ultrasound diagnosis system including an ultrasound observation device according to a first embodiment. As illustrated in FIG. 1, an ultrasound diagnosis system 1 includes: an ultrasound endoscope 2 that transmits ultrasound waves to a subject, which is the observation target, and receives ultrasound waves reflected by the subject; an ultrasound observation device 3 that generates ultrasound images based on ultrasound signals acquired by the ultrasound endoscope 2; a display device 4 that displays ultrasound images generated by the ultrasound observation device 3; and an input device 5 that receives inputs of command signals for setting a processing mode for an ultrasound image, setting an observation condition, and the like, for the ultrasound observation device 3.

The ultrasound endoscope 2 includes, at its distal end, an ultrasound transducer 21 that converts electric pulse signals received from the ultrasound observation device 3 into ultrasound pulses (sound pulses), emits them to the subject, converts ultrasound echoes reflected by the subject into electric echo signals (ultrasound signals) represented as changes in a voltage, and outputs them. The ultrasound transducer 21 may be any one of the radial type, the convex type, and the linear type. The ultrasound endoscope 2 may cause the ultrasound transducer 21 to conduct scanning mechanically or may cause it to conduct scanning electronically with elements arranged in array as the ultrasound transducer 21 by electronically switching elements for transmitting/receiving or by applying a delay to each element in transmitting/receiving.

The ultrasound endoscope 2 typically includes an optical imaging system and an imaging unit including an imaging element, and it is inserted into a digestive tract (esophagus, stomach, duodenum, large intestine) or respiratory apparatus (trachea, bronchi) of the subject so as to capture the digestive tract, respiratory apparatus, or their periphery organs (pancreas, gallbladder, bile duct, biliary tract, lymph node, mediastinal organ, blood vessel, or the like). Furthermore, the ultrasound endoscope 2 includes a light guide that guides illumination light emitted to the subject during capturing. The distal end of the light guide reaches the distal end of the insertion unit of the ultrasound endoscope 2 for the subject while the proximal end thereof is connected to a light source device that generates the illumination light.

The ultrasound observation device 3 includes a transmitting/receiving unit 31, a display controller 32, an input unit 33, a control determining unit 34, a control unit 35, and a storage unit 36. The ultrasound observation device 3 has set any one of the processing modes regarding multiple different processes performed on ultrasound images by a user and, based on a command signal output from the touch-pad described later, provides the function to perform the process corresponding to the set processing mode.

The transmitting/receiving unit 31 transmits and receives electric signals between the imaging unit and the ultrasound transducer 21. The transmitting/receiving unit 31 is electrically connected to the imaging unit so as to transmit capturing information, such as capturing timing, to the imaging unit and receive imaging signals generated by the imaging unit. Furthermore, the transmitting/receiving unit 31 is electrically connected to the ultrasound transducer 21 so as to transmit electric pulse signals to the ultrasound transducer 21 and receive echo signals, which are electrical reception signals, from the ultrasound transducer 21. Specifically, the transmitting/receiving unit 31 generates electric pulse signals based on the previously set waveform and transmission timing and transmits the generated pulse signals to the ultrasound transducer 21.

The transmitting/receiving unit 31 conducts STC (Sensitivity Time Control) compensation, or the like, to amplify echo signals having a larger receive depth with a higher amplification factor. The transmitting/receiving unit 31 performs processing such as filtering on amplified echo signals and then conducts A/D conversion, thereby generating and outputting digital high-frequency (RF: Radio Frequency) signals in time domain.

The display controller 32 generates endoscope image data based on imaging signals and ultrasound image data corresponding to electric echo signals. Furthermore, the display controller 32 superimposes various types of information on endoscope image data and ultrasound image data and outputs them, thereby controlling display on the display device 4. The display controller 32 is implemented by using a CPU (Central Processing Unit), various arithmetic circuits, or the like, having calculation and control functions.

The input unit 33 receives a command signal input through the input device 5 and receives input of various types of information corresponding to the received command signal. The various types of information include the setting of a processing mode for an ultrasound image, the setting of an observation condition (e.g., changing the gain and the display range), information on the touch position of a touching object on the touch-pad described later, and the like.

Based on the set processing mode, the control determining unit 34 determines the control to be performed out of a first control to stop a process on an ultrasound image and a second control to perform a predetermined process on an ultrasound image when it is detected that the touching object moves away from the touch-pad, described later, of the input device 5. The processing mode is, for example, the browsing mode for browsing ultrasound images arranged in chronological order by feeding them forward or backward, the rotation mode for rotating an ultrasound image displayed on the display device 4, the distance measurement mode for measuring the distance between any two points on an ultrasound image displayed on the display device 4, the region of interest (ROI) mode for changing the position or the size of the ROI that is set within an ultrasound image, and the like.

The control determining unit 34 determines that the first control is to be performed when the set processing mode is the processing mode (the distance measurement mode, the ROI mode) for performing a predetermined process on an ultrasound image by using information on the position at which the touching object moves away. Here, the distance measurement mode is a processing mode for performing a process by using information on the position at which the touching object moves away as it measures the distance between the touch start position and the touch end position. Furthermore, the ROI mode is a processing mode for performing a process by using information on the position at which the touching object moves away as it moves, enlarges, or reduces the ROI up to the touch end position.

The control determining unit 34 determines that the second control is to be performed when the set processing mode is the processing mode (the browsing mode, the rotation mode) for performing a predetermined process on an ultrasound image by using information on a movement at the touch position of the touching object. Here, the browsing mode is a processing mode for performing a process by using information on a movement at the touch position of the touching object as it feeds ultrasound images forward or backward in the direction in which an operation has been performed. Furthermore, the rotation mode is a processing mode for performing a process by using information on a movement at the touch position of the touching object as it rotates an ultrasound image clockwise or anticlockwise in the direction in which an operation has been performed.

The control determining unit 34 is implemented by using a CPU, various arithmetic circuits, or the like, having calculation and control functions.

The control unit 35 performs the overall control of the ultrasound diagnosis system 1. The control unit 35 is implemented by using a CPU, various arithmetic circuits, or the like, having calculation and control functions. The control unit 35 reads information, saved and stored in the storage unit 36, from the storage unit 36 and executes various arithmetic processes with regard to the method of operating the ultrasound observation device 3, thereby controlling the ultrasound observation device 3 in an integrated manner. Furthermore, the control unit 35 performs a process on an ultrasound image in accordance with determination by the control determining unit 34. Moreover, the control unit 35 may be configured by using the same CPU, or the like, as that of the display controller 32, the control determining unit 34, or the like.

The storage unit 36 stores data, and the like, including various programs for operating the ultrasound diagnosis system 1 and various parameters needed for processes of the ultrasound diagnosis system 1. The storage unit 36 stores, for example, the initial position (the sound ray number) of the write position of an ultrasound image (the transmission start position of an ultrasound wave).

Furthermore, the storage unit 36 stores various programs including an operation program for implementing the method of operating the ultrasound diagnosis system 1. The operation program may be widely distributed by being stored in a storage medium readable by a computer, such as hard disk, flash memory, CD-ROM, DVD-ROM, or flexible disk. Furthermore, the above-described various programs are available by being downloaded via a communication network. The communication network mentioned here is implemented by using, for example, an existing public network, LAN (Local Area Network), or WAN (Wide Area Network) regardless of whether it is wired or wireless.

The storage unit 36 having the above configuration is implemented by using a ROM (Read Only Memory) having various programs, and the like, previously installed therein, a RAM (Random Access Memory) storing calculation parameters, data, and the like, for processes, or the like.

The display device 4 is connected to the ultrasound observation device 3. The display device 4 is configured by using a display panel that is made of liquid crystal, organic EL (Electro Luminescence), or the like. The display device 4 displays, for example, ultrasound images output from the ultrasound observation device 3 and various types of information related to operations.

FIG. 2 is a diagram that illustrates a configuration of the input device illustrated in FIG. 1. As illustrated in FIG. 2, the external surface of the main body, which is a casing, of the input device 5 is coated with a cover made of silicone, or the like, with water-tightness. The input device 5 includes: a touch-pad 51 that detects touches with a touching object such as user's finger; and a display section 52 that is capable of displaying various types of information. The operating surface of the touch-pad 51 is, for example, square or rectangular. The input device 5 is electrically connected to the ultrasound observation device 3 via a cable, and it outputs signals, and the like, for command inputs for the touch-pad 51 to the input unit 33.

In the input device 5, when the touching object such as user's finger touches the touch-pad 51, the touch sensor detects the touch position and outputs a command signal to the ultrasound observation device 3. Furthermore, when the touching object moves while it is touching the touch-pad 51, the moving direction and the movement distance are detected and command signals are output to the ultrasound observation device 3. Based on received command signals, the ultrasound observation device 3 performs signal processing corresponding to the touch position and the moving direction and the movement distance of the touch position. Then, based on the received command signal, the ultrasound observation device 3 outputs an image having undergone processing by, for example, feeding an ultrasound image to be displayed on the display device 4 in chronological order, sliding or rotating the position of an ultrasound image, measuring a distance within an ultrasound image, or changing the position or the size of the ROI within the ultrasound image.

The display section 52 displays the setting of the processing mode, the setting of the observation condition, and the like. The display section 52 may be configured as a touch panel so as to change the setting of the processing mode, the setting of the observation condition, and the like.

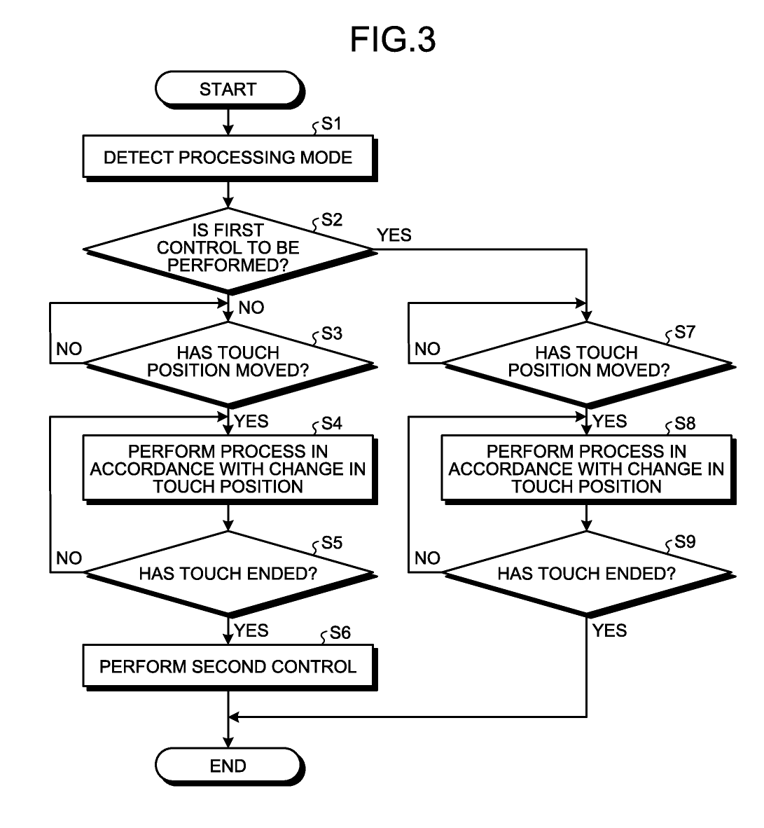

Next, a process of the ultrasound observation device 3 is explained in detail. FIG. 3 is a flowchart that illustrates a process of the ultrasound observation device according to the first embodiment. As illustrated in FIG. 3, after the touch-pad 51 is touched by a touching object and a process is started, the control unit 35 detects a processing mode (Step S1).

Then, the control determining unit 34 determines whether the first control is to be performed (Step S2). The control determining unit 34 determines that the first control is to be performed when the detected processing mode is the processing mode (the distance measurement mode, the ROI mode) for performing a predetermined process on an ultrasound image by using information on the position at which the touching object moves away. Conversely, the control determining unit 34 determines that the second control is to be performed when the set processing mode is the processing mode (the browsing mode, the rotation mode) for performing a predetermined process on an ultrasound image by using information on a movement at the touch position of the touching object.

When the control determining unit 34 determines that the first control is not to be performed (Step S2: No), that is, determines that the second control is to be performed, the control unit 35 determines whether the touch position of the touching object on the touch-pad 51 has moved based on a command signal received from the input unit 33 (Step S3).

When the control unit 35 determines that the touch position of the touching object on the touch-pad 51 has moved (Step S3: Yes), the display controller 32 performs a process on an ultrasound image in accordance with a change in the touch position of the touching object on the touch-pad 51 under the control of the control unit 35 (Step S4). Specifically, during the browsing mode, the display controller 32 performs the process to feed ultrasound images arranged in chronological order forward or backward in accordance with a change in the touch position of the touching object on the touch-pad 51. Furthermore, during the rotation mode, the display controller 32 performs the process to rotate the ultrasound image displayed on the display device 4 clockwise or anticlockwise in accordance with a change in the touch position of the touching object on the touch-pad 51.

Then, the control unit 35 determines whether a touch on the touch-pad 51 with the touching object has ended based on a command signal received from the input unit 33 (Step S5). When the control unit 35 determines that a touch on the touch-pad 51 with the touching object has ended (Step S5: Yes), the control unit 35 performs the second control based on a determination by the control determining unit 34 (Step S6). Specifically, at the same time as a touch on the touch-pad 51 with the touching object ends, the control unit 35 performs the process to feed an ultrasound image forward or backward in a state at a predetermined speed or the process to rotate an ultrasound image clockwise or anticlockwise in a state at a predetermined speed. Here, when the control unit 35 determines that a touch on the touch-pad 51 with the touching object continues (Step S5: No), the control unit 35 repeatedly performs the process at Step S4.

The control unit 35 may continue the second control until a predetermined termination command input is received after a touch on the touch-pad 51 with the touching object has ended or may automatically terminate the second control after a touch on the touch-pad 51 with the touching object has ended and a predetermined time has elapsed. Here, the termination command input may be a touch on the touch-pad 51 with the touching object or may be a press of a predetermined button.

At Step S2, when the control unit 35 performs the first control based on a determination by the control determining unit 34 (Step S2: Yes), the control unit 35 determines whether the touch position of the touching object on the touch-pad 51 has moved based on a command signal received from the input unit 33 (Step S7).

When the control unit 35 determines that the touch position of the touching object on the touch-pad 51 has moved (Step S7: Yes), the display controller 32 performs a process on an ultrasound image in accordance with a change in the touch position of the touching object on the touch-pad 51 under the control of the control unit 35 (Step S8). Specifically, in the distance measurement mode, the display controller 32 performs the process to measure the distance between the touch start position of the touching object on the touch-pad 51 and the current touch position, superimposes the distance on an ultrasound image, and causes the display device 4 to display it. Furthermore, in the ROI mode, the display controller 32 performs the process to change the position or the size of the ROI within the ultrasound image in accordance with a change in the touch position of the touching object on the touch-pad 51.

Then, the control unit 35 determines whether a touch on the touch-pad 51 with the touching object has ended based on a command signal received from the input unit 33 (Step S9). When the control unit 35 determines that a touch on the touch-pad 51 with the touching object has ended (Step S9: Yes), the control unit 35 performs a control to terminate the sequence of processes based on a determination by the control determining unit 34. Specifically, the control unit 35 performs, as the first control, a process to stop the process for measuring the distance on the ultrasound image or the process for changing the position or the size of the ROI within the ultrasound image at the same time as a touch on the touch-pad 51 with the touching object ends.

Thus, the sequence of processes is terminated, and a new process is started when there is a touch on the touch-pad 51 with the touching object again.

According to the first embodiment, as described above, in the browsing mode, ultrasound images are fed forward or backward in a state at a predetermined speed after an operation is finished; thus, the user is capable of sequentially browsing ultrasound images arranged in chronological order without performing operations. In this case, the setting for feeding ultrasound images at a high speed after the end of operation makes it possible to facilitate operation to feed several dozens to several hundreds of ultrasound images at once.

Similarly, in the rotation mode, an ultrasound image is rotated clockwise or anticlockwise in a state at a predetermined speed after an operation is finished; thus, the user is capable of rotating an ultrasound image to a desired position without performing operation. In this case, the setting for rotating an ultrasound image at a high speed after the end of operation makes it possible to facilitate operation to largely rotate an ultrasound image.

Conversely, in the distance measurement mode, a process is stopped after an operation is finished (that is, an extra process is not performed after an operation is finished) and therefore the user is capable of measuring the desired distance. Similarly, in the ROI mode, a process is stopped after an operation is finished, and therefore the user is capable of setting the ROI with the desired size and at the desired position.

In this manner, a process after the end of an operation is different depending on whether the set processing mode is the processing mode (the distance measurement mode, the ROI mode) for performing a predetermined process by using information on the position at which the touching object moves away or the processing mode (the browsing mode, the rotation mode) for performing a predetermined process by using information on a movement at the touch position of the touching object. As a result, in the browsing mode and the rotation mode, a desired process automatically continues without user's operation, whereby user's operation is simplified. Conversely, in the distance measurement mode and the ROI mode, when an operation is finished, the touch position at the end of the operation is determined, and the determined position is not shifted by the ultrasound observation device 3. Thus, the ultrasound observation device 3 is an ultrasound observation device that facilitates operation on the touch-pad.

### Second Embodiment

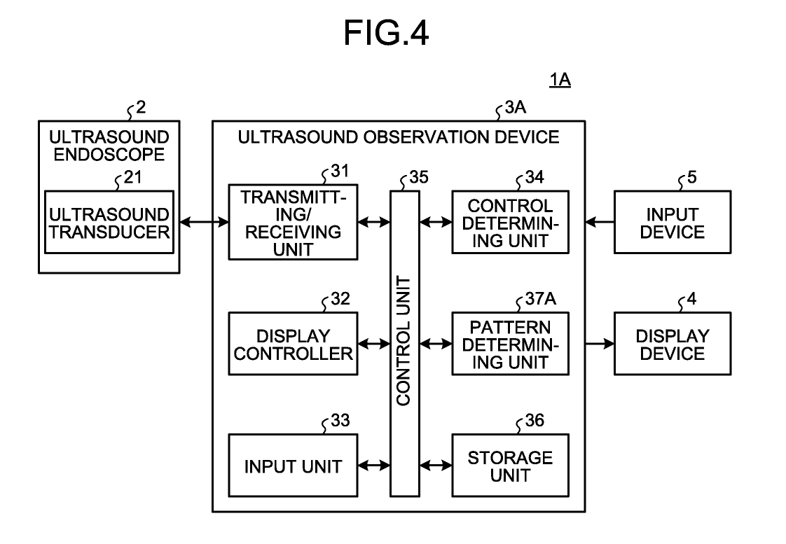

FIG. 4 is a block diagram that illustrates a configuration of an ultrasound diagnosis system including an ultrasound observation device according to a second embodiment. As illustrated in FIG. 4, an ultrasound observation device 3A in an ultrasound diagnosis system 1A includes a pattern determining unit 37A. As the other configurations are the same as those in the first embodiment, explanation is omitted.

The control determining unit 34 determines the control to be performed out of the first control and the second control based on the set processing mode and a determination result by the pattern determining unit 37A.

The pattern determining unit 37A determines whether a change in the touch position of the touching object on the touch-pad 51 is a predetermined operation pattern. Specifically, the pattern determining unit 37A determines whether a change in the touch position of the touching object is a predetermined operation pattern based on the speed of an operation by the touching object immediately before the touching object moves away from the touch-pad 51, the acceleration of an operation, the number of touches, the touch position, the pressing force due to the touching object against the touch-pad 51 (only when the touch-pad 51 is of a pressure-sensitive type), or the trajectory of the touch position of the touching object on the touch-pad 51.

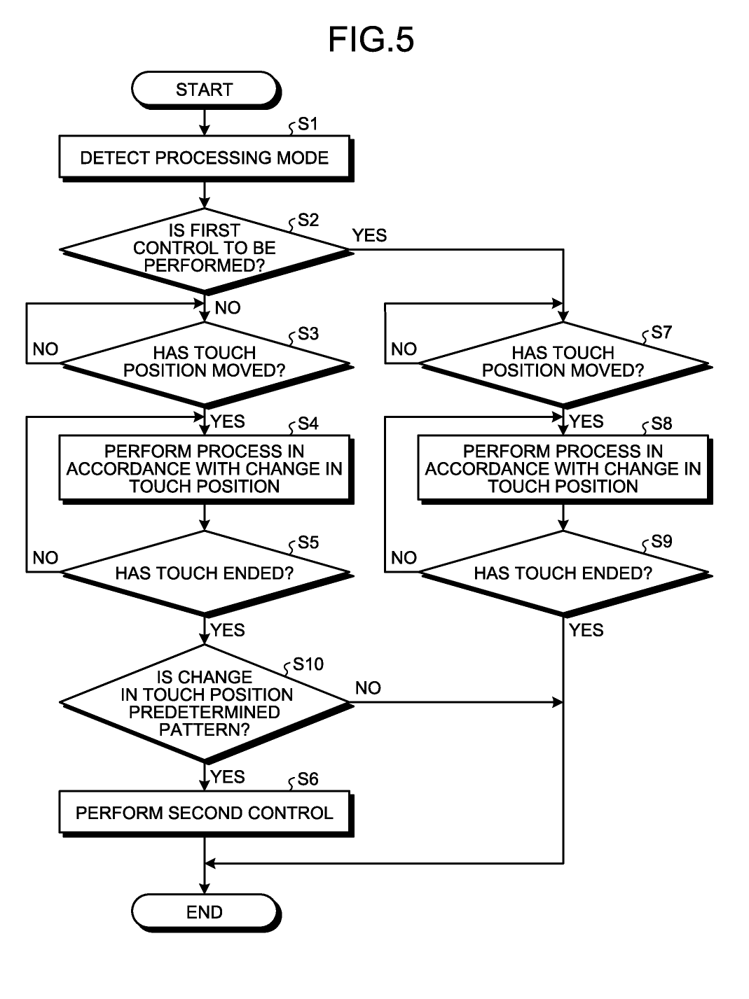

FIG. 5 is a flowchart that illustrates a process of the ultrasound observation device according to the second embodiment. As illustrated in FIG. 5, operations at Steps S1 to S5 are performed in the same manner as in the first embodiment.

Then, the pattern determining unit 37A determines whether a change in the touch position of the touching object on the touch-pad 51 is a predetermined operation pattern (Step S10). Specifically, the pattern determining unit 37A determines whether the speed (or acceleration) of an operation by the touching object immediately before the touching object moves away from the touch-pad 51 is higher than a predetermined threshold.

When the pattern determining unit 37A determines that a change in the touch position of the touching object on the touch-pad 51 is a predetermined operation pattern (Step S10: Yes), the control unit 35 performs the second control (Step S6). Specifically, the control unit 35 performs the process to feed ultrasound images forward or backward in a state at a predetermined speed or the process to rotate an ultrasound image clockwise or anticlockwise in a state at a predetermined speed at the same time as a touch on the touch-pad 51 with the touching object ends.

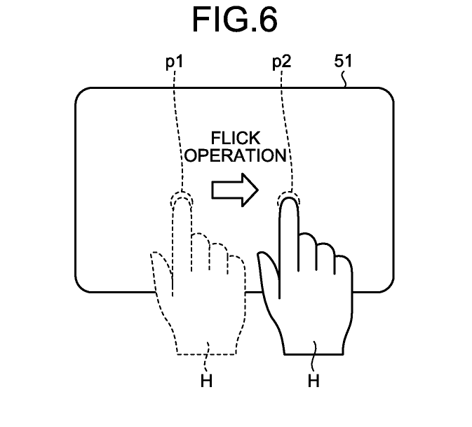

FIG. 6 is a diagram that illustrates a behavior for performing a flick operation on the touch-pad. As illustrated in FIG. 6, it is assumed that the touch position of the finger of the user's hand H on the touch-pad 51 is quickly moved from a position p1 to a position p2 so that a flick operation is performed, which is an operation in which the touch with the finger ends while the moving speed at the touch position is still fast. Here, the pattern determining unit 37A determines that it is a flick operation as the moving speed at the touch position immediately before the finger moves away from the touch-pad 51 is higher than a predetermined threshold. In other words, the pattern determining unit 37A determines that a change in the touch position of the touching object on the touch-pad 51 is a predetermined operation pattern (flick operation).

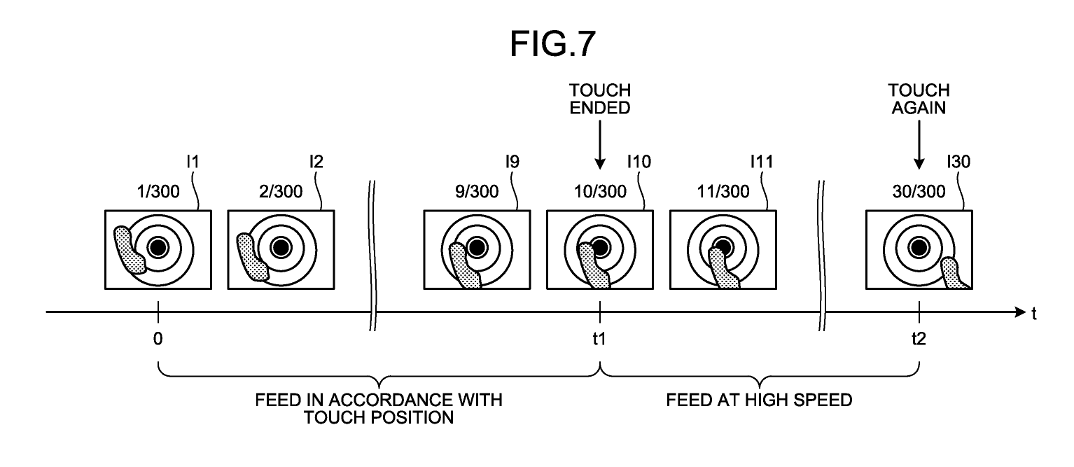

FIG. 7 is a diagram that illustrates a process when a flick operation is input to the touch-pad. As illustrated in FIG. 7, at the time t=0, the display device 4 displays an ultrasound image I1 that is the first ultrasound image among the ultrasound images arranged in chronological order.

Here, the total number of ultrasound images is 300, and the screen of the display device 4 displays the number (1/300, 2/300, . . . ) indicating the sequential order of an ultrasound image. Then, after the touch on the touch-pad 51 with the finger starts at the time t=0 and the touch position of the finger starts to move, the ultrasound image displayed on the display device 4 is moved from the ultrasound image I1 to a second ultrasound image 12. Further, it is assumed that, while the touch position with the finger moves, ultrasound images are sequentially fed in accordance with the touch position, a ninth ultrasound image 19 is moved, and the touch with the finger ends at the time t=t1 when a tenth ultrasound image I10 is displayed. In the case of a flick operation, the pattern determining unit 37A determines that a change in the touch position of the touching object on the touch-pad 51 is a predetermined operation pattern (flick operation), the control unit 35 performs the process to feed ultrasound images in a state at a predetermined speed (the second control). For example, the control unit 35 performs the process to feed ultrasound images at a high speed, starting from an eleventh ultrasound image I11. As a result, the user is capable of feeding several dozens to several hundreds of ultrasound images at once without performing any operations. Furthermore, if the touch-pad 51 is touched again with the finger at the time t=t2 when a thirtieth ultrasound image 130 is displayed, the process to feed ultrasound images is stopped at that time. In this manner, the control determining unit 34 may determine that the second control is to be stopped when it is detected that the touch-pad 51 is touched by the touching object while the control unit 35 is performing the second control.

Conversely, at Step S10, when the pattern determining unit 37A determines that a change in the touch position of the touching object on the touch-pad 51 is not a predetermined operation pattern (Step S10: No), the control unit 35 performs a control to terminate the sequence of processes. Specifically, the control unit 35 stops, as the first control, the process for feeding ultrasound images forward or backward or the process for rotating an ultrasound image clockwise or anticlockwise at the same time as the touch on the touch-pad 51 with the touching object ends.

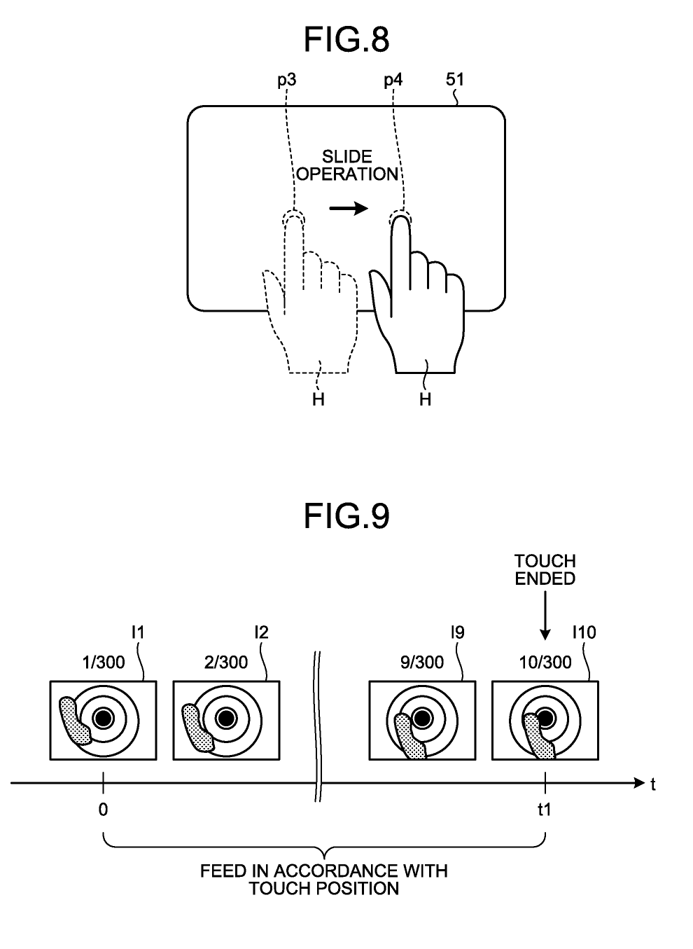

FIG. 8 is a diagram that illustrates a behavior for performing a slide operation on the touch-pad. As illustrated in FIG. 8, it is assumed that the touch position of the finger of the user's hand H on the touch-pad 51 is slowly moved from a position p3 to a position p4 so that a slide operation is performed, which is an operation in which the touch with the finger ends while the moving speed at the touch position is slow. Here, the pattern determining unit 37A determines that it is a slide operation as the moving speed at the touch position immediately before the finger moves away from the touch-pad 51 is less than a predetermined threshold. In other words, the pattern determining unit 37A determines that a change in the touch position of the touching object on the touch-pad 51 is not a predetermined operation pattern (flick operation).

FIG. 9 is a diagram that illustrates a process when a slide operation is input to the touch-pad. As illustrated in FIG. 9, at the time t=0 to t1, ultrasound images are fed in accordance with the touch position in the same manner as in FIG. 7. Then, in the case of a slide operation, at the time t=t1 when the touch with the finger ends, the pattern determining unit 37A determines that a change in the touch position of the touching object on the touch-pad 51 is not a predetermined operation pattern (flick operation), and therefore the control unit 35 performs the process (the first control) to stop the process for feeding ultrasound images. That is, the state where the ultrasound image I10 is presented on the display device 4 is maintained.

As described above, according to the second embodiment, the process on an ultrasound image after the touch with the touching object, such as finger, ends is different depending on a processing mode and a determination result by the pattern determining unit 37A. As a result, in the browsing mode or the rotation mode, the user is capable of differently using the first control and the second control depending on an operation. Specifically, in the browsing mode, the user is capable of feeding (finely adjusting) any number of ultrasound images by performing a slide operation. Furthermore, in the browsing mode, the user is capable of feeding (coarsely adjusting) ultrasound images at a high speed by performing a flick operation. Similarly, in the rotation mode, the user is capable of rotating (finely adjusting) an ultrasound image at any small angle by performing a slide operation. Furthermore, in the rotation mode, the user is capable of rotating (coarsely adjusting) an ultrasound image at a high speed by performing a flick operation.

Furthermore, in the distance measurement mode and the ROI mode, even when the user performs either slide operation or flick operation, the process on an ultrasound image is stopped at the end of the operation; therefore, when an operation is finished, the touch position at the end of the operation is determined. Thus, the ultrasound observation device 3A is an ultrasound observation device that further facilitates operation on the touch-pad as compared with the ultrasound observation device 3 according to the first embodiment.

Furthermore, the pattern determining unit 37A may determine whether the number of touches with the touching object immediately before the finger moves away from the touch-pad 51 is more than a predetermined threshold. For example, the pattern determining unit 37A determines whether the touch-pad 51 is operated with two or more fingers. Furthermore, the pattern determining unit 37A may determine whether the touch position of the touching object immediately before the finger moves away from the touch-pad 51 is included in a predetermined area. Furthermore, the pattern determining unit 37A may determine whether the pressing force due to the touching object against the touch-pad 51 immediately before the finger moves away from the touch-pad 51 is more than a predetermined threshold. Moreover, the pattern determining unit 37A may determine whether a change in the touch position of the touching object is a predetermined operation pattern in accordance with the trajectory of the touch position of the touching object on the touch-pad 51. For example, the pattern determining unit 37A determines that a change in the touch position of the touching object is a predetermined operation pattern when the trajectory of the touch position of the touching object on the touch-pad is circular. Here, the control unit 35 rotates an ultrasound image in a state at a predetermined speed as, for example, the second control.

Furthermore, the control determining unit 34 may determine that, as the second control, the process is performed to feed ultrasound images forward or backward by gradually increasing the speed when the pattern determining unit 37A determines that a change in the touch position of the touching object is a predetermined operation pattern multiple times in a row. As a specific example, the control determining unit 34 performs, as the second control, the process to feed ultrasound images forward or backward in a state at a predetermined speed when the pattern determining unit 37A determines that a change in the touch position of the touching object is a predetermined operation pattern for the first time, and it performs, as the second control, the process to feed ultrasound images forward or backward in a state at a speed twice as high as the predetermined speed when the pattern determining unit 37A determines that a change in the touch position of the touching object is a predetermined operation pattern for the second time in a row.

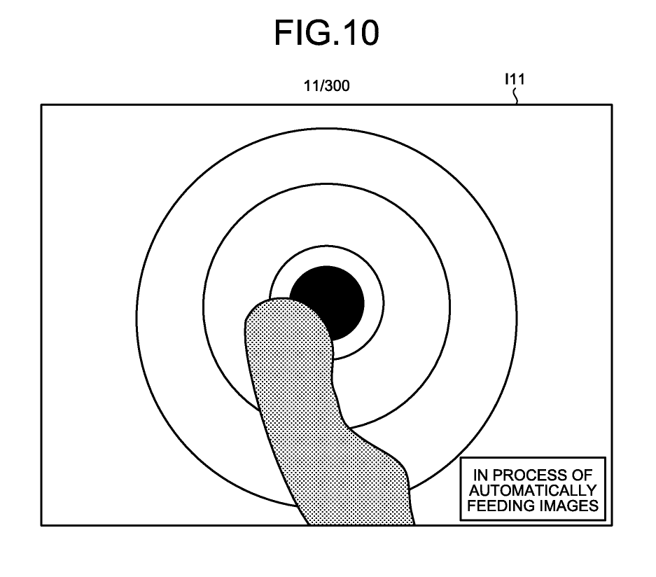

FIG. 10 is a diagram that illustrates an example of the screen when the second control is being performed. As illustrated in FIG. 10, the display controller 32 may cause the display device 4 to indicate that the process (the second control) for feeding ultrasound images forward or backward in a state at a predetermined speed is being performed by using texts such as “in the process of automatically feeding images”, a predetermined indicator, or the like. Furthermore, in this manner, the display controller 32 may cause the display device 4 to indicate which processing mode is the currently set processing mode, which one of the first control and the second control is to be performed when it is detected that the touching object moves away from the touch-pad 51, either the first control or the second control is being performed, or the like. Furthermore, the display controller 32 may notify a user of the above information by using a way such as sound or vibration.

In the example explained according to the above-described embodiment, at Step S11, the control determining unit 34 determines that, as the second control, the process is performed to feed ultrasound images forward or backward in a state at a predetermined speed at the same time as the touch on the touch-pad 51 with the touching object ends; however, this is not a limitation. At the same time as the touch on the touch-pad 51 with the touching object ends, the control determining unit 34 may determine that, as the second control, the process is performed to feed ultrasound images forward or backward in a state at the speed corresponding to the moving speed at the touch position immediately before the touch ends. Specifically, the control determining unit 34 determines that a process is performed to feed ultrasound images forward or backward in a state at the speed corresponding to the moving speed at the last touch position, feed ultrasound images forward or backward for the number of times (by the number of pieces) corresponding to the moving speed at the last touch position, feed ultrasound images forward or backward by the time corresponding to the moving speed at the last touch position, or the like. Similarly, the control determining unit 34 may determine that, as the second control, a process is performed in accordance with the number of touches with the touching object immediately before the touching object moves away from the touch-pad 51, the touch position, the pressing force against the touch-pad 51, or the trajectory of the touch position of the touching object on the touch-pad 51. Furthermore, the control determining unit 34 may determine that, as the second control, a process is performed based on the setting made by the user or an automatically learned calculation result.

Furthermore, in the example described according to the above embodiment, the control determining unit 34 determines that the first control is to be performed when the distance measurement mode or the ROI mode is set and the second control is to be performed when the browsing mode or the rotation mode is set; however, this is not a limitation. For example, the control determining unit 34 determines that the second control is to be performed when the ROI mode is set, and the control unit 35 performs, as the second control, the process to change the position or the size of the ROI within an ultrasound image little by little at the same time as the touch on the touch-pad 51 with the touching object ends. As a result, users are capable of finely adjusting the position or the size of the ROI at such a level that manual operation is difficult.

According to the present disclosure, it is possible to provide an ultrasound observation device, a method of operating the ultrasound observation device, and a program for operating the ultrasound observation device that facilitate operation on a touch-pad.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An ultrasound observation device comprising a processor comprising hardware, the processor being configured to execute:
setting any one of processing modes regarding multiple different processes performed on an ultrasound image by a user;
performing, based on a command signal output from a touch-pad in accordance with a change in a touch position of a touching object, a process corresponding to the set processing mode, the ultrasound observation device; and
determine, based on the set processing mode, a control to be performed out of a first control to stop a process on the ultrasound image and a second control to perform a predetermined process on the ultrasound image when it is detected that the touching object moves away from the touch-pad.

2. The ultrasound observation device according to claim 1, wherein the processor further
determines whether a change in a touch position of the touching object on the touch-pad is a predetermined operation pattern, and
determines a control to be performed out of the first control and the second control based on the set processing mode and a determination result of whether the change in the touch position of the touching object on the touch-pad is the predetermined operation pattern.

3. The ultrasound observation device according to claim 2, wherein the processor determines whether the change in the touch position of the touching object is the predetermined operation pattern based on a speed of an operation with the touching object immediately before the touching object moves away from the touch-pad, an acceleration of an operation, a number of touches, a touch position, a pressing force due to the touching object against the touch-pad, or a trajectory of the touch position of the touching object on the touch-pad.

4. The ultrasound observation device according to claim 1, wherein the processor determines that the first control is to be performed when the set processing mode is a processing mode for performing the predetermined process on the ultrasound image by using information on a position at which the touching object moves away from the touch-pad.

5. The ultrasound observation device according to claim 1, wherein the processor determines that the second control is to be performed when the set processing mode is a processing mode for performing the predetermined process on the ultrasound image by using information on a movement at the touch position of the touching object on the touch-pad.

6. The ultrasound observation device according to claim 1, wherein the processor determines that, as the predetermined process, a process is performed to feed the ultrasound images forward or backward in a state at a predetermined speed when the set processing mode is a browsing mode for browsing the ultrasound images arranged in chronological order while feeding the ultrasound images forward or backward and when it is detected that the touching object moves away from the touch-pad.

7. The ultrasound observation device according to claim 1, wherein the processor determines that, as the predetermined process, a process is performed to rotate the ultrasound image clockwise or anticlockwise in a state at a predetermined speed when the set processing mode is a rotation mode for rotating the ultrasound image displayed on a display device and when it is detected that the touching object moves away from the touch-pad.

8. The ultrasound observation device according to claim 1, further comprising a controller configured to perform a process on the ultrasound image in accordance with a determination by the processor, wherein
the processor determines that the control is performed to stop the second control when it is detected that the touch-pad is touched by the touching object while the controller is performing the process for the second control.

9. The ultrasound observation device according to claim 1, further comprising a display controller configured to cause a display to display the set processing mode, a determination by the processor, or the process being performed by the ultrasound observation device.

10. A method of operating an ultrasound observation device, the method comprising:
setting any one of processing modes regarding multiple different processes performed on an ultrasound image by a user;
performing, based on a command signal output from a touch-pad in accordance with a change in a touch position of a touching object, a process corresponding to the set processing mode; and
determining, based on the set processing mode, a control to be performed out of a first control to stop a process on the ultrasound image and a second control to perform a predetermined process on the ultrasound image when it is detected that the touching object moves away from the touch-pad.

11. A non-transitory computer-readable recording medium on which an executable program is recorded, the program causes a processor of an ultrasound observation device to execute:
setting any one of processing modes regarding multiple different processes performed on an ultrasound image by a user;
performing, based on a command signal output from a touch-pad in accordance with a change in a touch position of a touching object, a process corresponding to the set processing mode; and
determining, based on the set processing mode, a control to be performed out of a first control to stop a process on the ultrasound image and a second control to perform a predetermined process on the ultrasound image when it is detected that the touching object moves away from the touch-pad.
